(19) United States
(12) Patent Application Publication
(10) Pub. No.: US 2023/0137823 A1
(43) Pub. Date: May 4, 2023

(54) KITS AND METHODS FOR ENRICHMENT OF FULL-LENGTH RNA

(71) Applicant: Eclipse Bioinnovations, Inc., San Diego, CA (US)

(72) Inventors: DANIEL A. LORENZ, San Diego, CA (US); Anide Johansen, San Diego, CA (US); Karen B. Chapman, San Diego, CA (US)

(21) Appl. No.: 17/975,259

(22) Filed: Oct. 27, 2022

Related U.S. Application Data
(60) Provisional application No. 63/273,001, filed on Oct. 28, 2021.

(51) Int. Cl. C12N 15/10; C12Q 1/6806; C12Q 1/6855

(52) U.S. Cl. CPC C12N 15/1096; C12Q 1/6806; C12Q 1/6855; C12N 15/1093

(57) ABSTRACT

Described herein are, at least in part, novel methods for enriching full length RNAs. In aspects of the disclosure, methods for enrichment of RNAs, kits for making such full length RNA reads, and composition for enriching RNAs or cDNA libraries derived therefrom are provided herein. In some embodiments, the method for enriching for full length RNA comprises isolating an RNA sample, contacting the RNA sample with a 5′ exonuclease; and performing a reverse transcription reaction to convert the RNA to cDNA.

### CROSS-REFERENCE TO RELATED APPLICATIONS

This application claims the benefit of U.S. Provisional Application No. 63/273,001 filed on Oct. 28, 2021, which is incorporated by reference in its entirety.

### BACKGROUND

Sequencing of RNA transcripts has been essential to our understanding of biology. Recent advancements in sequencing technologies, generally termed third-generation sequencing technologies, have enabled sequencing longer, sometimes even full-length RNA transcripts. One major advantage of these longer reads is the ability to unambiguously assign transcript isoforms. However, this assignment is often limited to full-length reads as isoforms often vary at their 5′ and 3′ ends.

While these third-generation technologies enable full-length sequencing they do not select for intact RNA molecules, thus potentially wasting a large fraction of their sequencing reads on incomplete data.

### SUMMARY

Some embodiments of the present disclosure relate to a method of selecting for full-length RNA molecules. The method may include isolating total RNA from a sample, removing uncapped RNA molecules with a 5′ exonuclease, and then selecting any of the remaining capped RNA molecules for those RNA with a polyadenylated tail.

Some embodiments of the present disclosure relate to a method of selecting for full-length RNA molecules. The method comprises: 1) isolating total RNA, 2) removal of uncapped RNA molecules with a 5′ exonuclease, 3) select for polyadenylated RNA and 4) generate a direct RNA sequencing library for an Oxford Nanopores® direct RNA sequencing platform.

Some embodiments of the present disclosure relate to a method of selecting for full-length RNA molecules. The method comprises: 1) isolating total RNA, 2) removal of uncapped RNA molecules with a 5′ exonuclease, 3) perform a reverse transcription reaction to convert the RNA to cDNA using an oligodT primer, 4) use the cDNA to prepare an RNA sequencing library for third-generation sequencing platforms such as those offered by Oxford Nanopore Technologies and Pacific Biosciences.

Some embodiments relate to a method of enriching for full length RNA. In some embodiments, the method comprises contacting an RNA sample with a 5′ exonuclease, and selecting for 3′ poly A tails. In some embodiments, the poly A tail selection occurs by templated ligation, a poly A complementary reverse transcription, or poly A based hybridization capture. In some embodiments, an alternative 5′ cap selection method is used, including the use of cap recognizing antibodies for immunoprecipitation and dephosphorylation of non-capped RNAs followed by cap removal. In some embodiments, the RNA sample is selected from the group consisting of cells, tissue, FFPE, blood, urine, and saliva. In some embodiments, the RNA sample is total cellular RNA. In some embodiments, the RNA sample comprises 5 μg or less of RNA. In some embodiments, the RNA sample is less than 50 ng RNA. In some embodiments, the RNA sample comprises degraded RNA. In some embodiments, the RNA sample is partially degraded. In some embodiments, the RNA sample comprises mRNA molecules. In some embodiments, RNA sample is rRNA depleted. In some embodiments, the RNA sample comprises capped RNA. In some embodiments, the RNA sample enriched by the method is full length eukaryotic mRNA. In some embodiments, the method further comprises sequencing the full length RNA. In some embodiments, the method further comprises sequencing the cDNA. In some embodiments, the RNA sample has a starting RIN score of less than 7. In some embodiments, sequencing of the cDNA is further subjected to a size selection procedure to produce a cDNA library. In some embodiments, the method further comprises amplifying the cDNA library by third generation sequencing technologies. In some embodiments, the RNA sample is between about 50 nucleotides and about 5000 nucleotides. In some embodiments, the 5′ exonuclease is Terminator or XRN-1.

Some embodiments relate to method of enriching for full length RNA. The method comprises isolating an RNA sample, contacting the RNA sample with a 5′ exonuclease, selecting for 3′ poly A tails, and performing a reverse transcription reaction to convert the RNA to cDNA, thereby enriching for capped RNA for full length reads. In some embodiments, the poly A tail selection occurs by templated ligation, a poly A complementary reverse transcription, or poly A based hybridization capture. In some embodiments, an alternative 5′ cap selection method is used, including the use of cap recognizing antibodies for immunoprecipitation and dephosphorylation of non-capped RNAs followed by cap removal. In some embodiments, the RNA sample is selected from the group consisting of cells, tissue, FFPE, blood, urine, and saliva. In some embodiments, the RNA sample is total cellular RNA. In some embodiments, the RNA sample comprises 5 μg or less of RNA. In some embodiments, the RNA sample comprises degraded RNA. In some embodiments, the RNA sample has a starting RIN score of less than 7. In some embodiments, the RNA sample is less than 50 ng RNA. In some embodiments, the RNA sample is partially degraded. In some embodiments, the RNA sample comprises mRNA molecules. In some embodiments, RNA sample is rRNA depleted. In some embodiments, the RNA sample comprises capped RNA. In some embodiments, the RNA sample enriched by the method is full length eukaryotic mRNA. In some embodiments, the method further comprises sequencing the full length RNA. In some embodiments, the method further comprises sequencing the cDNA. In some embodiments, sequencing of the cDNA is further subjected to a size selection procedure to produce a cDNA library. In some embodiments, the method further comprises amplifying the cDNA library by third generation sequencing technologies. In some embodiments, the RNA sample is between about 50 nucleotides and about 10,000 nucleotides. In some embodiments, the 5′ exonuclease is Terminator or XRN-1. In some embodiments, the method improves yields of low quality RNA by at least 70%.

Some embodiments relate to a kit. In some embodiments, the kit comprises a first component comprising a 5′ exonuclease, and a second component comprising reverse transcription reaction materials comprising buffer, primers, dNTP mix, and a reverse transcriptase, and a manual providing instructions for enriching full length RNA. In some embodiments, the kit further comprises unconjugated oligos, RNA cleanup columns, and kinases.

These and other features, aspects, and advantages of the present disclosure will become better understood with reference to the following description and appended claims.

### BRIEF DESCRIPTION OF THE DRAWINGS

FIG. 1 illustrates a schematic diagram depicting one embodiment of a protocol for the enrichment of full length RNA molecules.

FIG. 2A illustrates a barplot depicting improved yields from low RIN samples using ONT cDNA library prep quantified from the corresponding tapestation using Terminator exonuclease.

FIG. 2B illustrates a tapestation of the samples from the barplot of FIG. 2A.

FIG. 3A illustrates a barplot depiciting improved yields from low RIN samples using XRN-1 following PacBio library preparation

FIG. 3B illustrates a tapestation of the samples from the barplot of FIG. 3B.

FIG. 4 illustrates a genome track view of RPS2 showing improved coverage across the gene with Terminator exonuclease treatment with ONT cDNA library preperation from FIG. 1.

FIG. 5 illustrates a transcriptome wide metagene plot of the 5′ end of reads with ONT cDNA library preperation from FIG. 1.

FIG. 6A illustrates a barplot depicting improved yields from low RIN samples using PacBio Isoseq library prep quantified from the corresponding tapestation using Terminator exonuclease.

FIG. 6B illustrates a tapestation of the samples shown in FIG. 6A.

FIG. 7 illustrates a genome track view of RPS2 showing improved coverage across the gene with Terminator exonuclease treatment with PacBio Isoseq library preperation from FIG. 6A.

FIG. 8 illustrates a transcriptome wide metagene plot of the 5′ end of reads with PacBio Isoseq library preperation from FIG. 6A.

### DETAILED DESCRIPTION

In the Summary Section above and the Detailed Description Section, and the claims below, reference is made to particular features of the disclosure. It is to be understood that the disclosure of the disclosure in this specification includes all possible combinations of such particular features. For example, where a particular feature is disclosed in the context of a particular aspect or embodiment of the disclosure, or a particular claim, that feature can also be used, to the extent possible, in combination with and/or in the context of other particular aspects and embodiments of the disclosure, and in the disclosure generally.

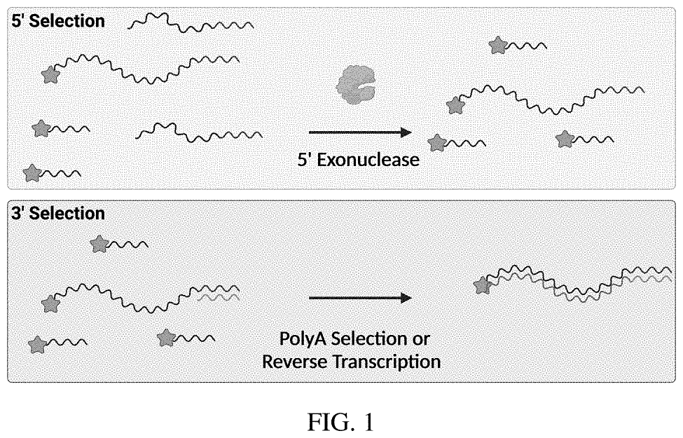

One embodiment as shown in FIG. 1 is a method for enriching a population of mRNA molecules in a mixture of RNAs to select for full-length messenger RNAs which typically contain a 5′ cap and a 3′ polyadenylation. The method may include obtaining a sample containing RNA from a biological source. The sample may contain RNA with a reduce quality such that a relatively large proportion of the RNA in the sample has been degraded. To enrich for full-length mRNA the sample may be first contacted by a 5′ exonuclease which digest RNA which does not have a 5′ cap. After uncapped RNA has been digested, the RNA in the sample may then be selected for only those RNA molecules which have a 3′ polyadenylation. This can be done by binding the RNA to oligodT primers in solution or on a substrate. Once the oligodT primers have been bound, the unbound RNA may be removed, or the primers may be used with reverse transcriptase to generate cDNA from the full-length RNA. The only RNA in the sample with both a 5′ cap and the polyadenylation site should be the full-length RNA. Thus, this process, and any kit which enables this process, may be used to obtain and enrich for full-length RNA is a sample containing degraded RNA.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

As used herein, the articles “a”, “an”, and “the” relate equivalently to a meaning as singular or plural unless the context dictates otherwise.

As used herein and as conventionally understood by those in the relevant art, a “nucleotide” comprises a base, a sugar and one or more phosphate groups. The base (also referred to as a “nitrogenous base” or a “nucleobase”) is typically a purine or pyrimidine. The sugar is typically a five-carbon ribose (as in ribonucleotides) or a 2-deoxyribose (as in deoxyribonucleotides), which is bound via a glycosidic linkage to the base. Nucleotides typically have one, two or three phosphate groups (mono-, di- or tri-phosphates). Generally, the phosphate groups form a chemical bond at the 5-carbon position of the sugar, although they can also bond at the 2 or 3-carbon positions of the sugar group. Cyclic nucleotides form when a phosphate group is bound to two hydroxyl groups on the sugar. A “nucleoside” comprises a nucleobase and sugar. A nucleotide can thus also be called a nucleoside mono-, di- or triphosphate.

As used herein, “nucleic acid” refers to DNA, RNA and derivatives thereof. The terms “DNA” and “RNA” refer to deoxyribonucleic acid and ribonucleic acid, respectively. The term “mRNA” refers to messenger RNA. The term rRNA refers to ribosomal RNA. The term “tRNA” refers to transfer RNA.

As used herein, “full-length RNA” is any RNA molecule that is intact. Defined by starting from its 5′ transcriptional start site and ending with its 3′ transcriptional stop site.

As used herein, “cap” or “capped” with respect to RNAs refers to the cap found on the 5′ end of an mRNA molecule which consists of a guanine nucleotide connected to the mRNA via an unusual 5′ to 5′ triphosphate linkage. This guanosine is methylated on the 7 position directly after capping in vivo by a methyl transferase. It is also referred to as a 7-methylguanylate cap, abbreviated m 7 G or m 7 Gppp. Capped RNA refers to an RNA comprising a 5′ cap and is also referred to as Gppp-RNA.

As used herein, the term “MicroRNA” (miRNA) refers to RNA molecules that are processed from small hairpin RNA (shRNA) precursors that are produced from miRNA genes. miRNAs are 21-23 nucleotides in length and through the RNA-induced silencing complex they target and silence mRNAs containing imperfectly complementary sequence. Animal miRNAs are initially transcribed as part of one arm of an 80 nucleotide RNA stem-loop that in turn forms part of a several hundred nucleotides long miRNA precursor termed a primary miRNA (pri-miRNA). Animal miRNAs are initially transcribed as pri-miRNA by DNA-dependent RNA polymerase II, then processed by Drosha as pre-miRNA, a stem-loop structure molecule, and finally cut by Dicer to form mature miRNA.

Any protein described herein may be non-naturally occurring, where the term “non-naturally occurring” refers to a protein that has an amino acid sequence and/or a post-translational modification pattern that is different to the protein in its natural state. For example, a non-naturally occurring protein may have one or more amino acid substitutions, deletions or insertions at the N-terminus, the C-terminus and/or between the N- and C-termini of the protein. A “non-naturally occurring” protein may have an amino acid sequence that is different to a naturally occurring amino acid sequence (i.e., having less than 100% sequence identity to the amino acid sequence of a naturally occurring protein) but that that is at least 80%, at least 85%, at least 90%, at least 95%, at least 97%, at least 98% or at least 99% identical to the naturally occurring amino acid sequence. In certain cases, a non-naturally occurring protein may contain an N-terminal methionine or may lack one or more post-translational modifications (e.g., glycosylation, phosphorylation, etc.) if it is produced by a different (e.g., bacterial) cell. A “mutant” protein may have one or more amino acid substitutions relative to a wild-type protein and may include a “fusion” protein. The term “fusion protein” refers to a protein composed of a plurality of polypeptide components that are unjoined in their native state. Fusion proteins may be a combination of two, three or even four or more different proteins. The term polypeptide includes fusion proteins, including, but not limited to, a fusion of two or more heterologous amino acid sequences, a fusion of a polypeptide with: a heterologous targeting sequence, a linker, an immunologically tag, a detectable fusion partner, such as a fluorescent protein, β-galactosidase, luciferase, etc., and the like. A fusion protein may have one or more heterologous domains added to the N-terminus, C-terminus, and or the middle portion of the protein. If two parts of a fusion protein are “heterologous”, they are not part of the same protein in its natural state.

In the context of a nucleic acid, the term “non-naturally occurring” refers to a nucleic acid that contains: a) a sequence of nucleotides that is different to a nucleic acid in its natural state (i.e. having less than 100% sequence identity to a naturally occurring nucleic acid sequence), b) one or more non-naturally occurring nucleotide monomers (which may result in a non-natural backbone or sugar that is not G, A, T or C) and/or c) may contain one or more other modifications (e.g., an added label or other moiety) to the 5′-end, the 3′ end, and/or between the 5′- and 3′-ends of the nucleic acid.

In the context of a preparation, the term “non-naturally occurring” refers to: a) a combination of components that are not combined by nature, e.g., because they are at different locations, in different cells or different cell compartments; b) a combination of components that have relative concentrations that are not found in nature; c) a combination that lacks something that is usually associated with one of the components in nature; d) a combination that is in a form that is not found in nature, e.g., dried, freeze dried, crystalline, aqueous; and/or e) a combination that contains a component that is not found in nature. For example, a preparation may contain a “non-naturally occurring” buffering agent (e.g., Tris, HEPES, TAPS, MOPS, tricine or MES), a detergent, a dye, a reaction enhancer or inhibitor, an oxidizing agent, a reducing agent, a solvent or a preservative that is not found in nature.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

All references cited herein, including but not limited to published and unpublished applications, patents, and literature references, are incorporated herein by reference in their entirety and are hereby made a part of this specification.

Where a range of values is provided, it is understood that the upper and lower limit, and each intervening value between the upper and lower limit of the range is encompassed within the embodiments.

### Methods and Uses

In aspects, the disclosure relates to a method of enriching a full length RNA molecule as shown in FIG. 1. Some embodiments of the present disclosure relate to a method of selecting for full-length RNA molecules. In some embodiments, the method includes isolating total RNA, removal of uncapped RNA molecules with a 5′ exonuclease.

Some embodiments of the present disclosure relate to a method of selecting for full-length RNA molecules. In some embodiments, the method includes isolating total RNA, removal of uncapped RNA molecules with a 5′ exonuclease, and generating a direct RNA sequencing library for direct RNA sequencing. In some embodiments, the direct RNA sequencing may be performed on an RNA sequencing platform. In some embodiments, the RNA sequencing platform may be Oxford Nanopore direct RNA sequencing platform.

Some embodiments of the present disclosure relate to a method of selecting for full-length RNA molecules. In some embodiments, the method includes isolating total RNA, removing uncapped RNA molecules with a 5′ exonuclease, performing a reverse transcription reaction to convert the RNA to cDNA, and using the cDNA to prepare an RNA sequencing library.

In some embodiments, the method further comprises isolating a sample. In some embodiments, the sample includes (or contains) RNA. In some embodiments, the starting RNA sample includes total cellular RNA (e.g., structural RNA (such as rRNA and tRNA as well as mRNA) and includes both polyA-i-RNA and polyA-RNA. In some embodiments, RNA may be obtained from a cell. For example, the cell is lysed and a starting RNA sample is obtained using known RNA isolation techniques. Exemplary methods include, for example, a MasterPure™ RNA Purification Kit, an ArrayPure™ Nano-Scale RNA.

In some embodiments, the method comprises a purification step. In some embodiments, the purification step utilizes a purification kit from a commercial source or using a “homebrew” method known in the art. Alternatively, in some embodiments, a sample including or containing RNA can contain a subtraction of total RNA obtained by any method known in the art, such as but without limitation, a subfraction based on size (e.g., by purification on an agarose or polyacrylamide gel, or by column purification, including by HPLC), or a subfraction obtained by salt precipitation (e.g., using precipitation with 0.5-2.5 M LiCl (Barlow et al., Biochem. Biophys. Res. Comm. 13:61, 1963); Cathala et al, DNA 2: 329, 1983) or 2.5 M ammonium acetate).

In some embodiments, a sample comprising RNA can also contain DNA. In some embodiments, the method includes a step for enriching an RNA having a 5′-cap in a biological sample comprising prokaryotic RNA, eukaryotic RNA or both prokaryotic and eukaryotic RNA and at least one undesired nucleic acid, e.g., structural RNA.

In some embodiments, the RNA having a 5′-cap is selected from, but not limited to, the group comprising: (i) prokaryotic mRNA; (ii) eukaryotic mRNA, including polyadenylated and non-polyadenylated eukaryotic mRNA; (iii) a mixture of both prokaryotic and eukaryotic mRNA; (iv) eukaryotic snRNA; (v) eukaryotic pre-micro RNA; and (vi) prokaryotic or eukaryotic primary RNA transcripts of unknown function. In some embodiments, the RNA is rRNA depleted.

In one embodiment, a starting RNA population for use in the methods described herein include mRNA as well as other capped but less abundant pri-miRNA and/or pri-piRNAs or other non-coding regulatory RNAs. In some embodiments, the RNA in the preparation includes a naturally capped RNA and prior to performing the methods described herein. In some embodiments, a capping enzyme is added to an RNA sample. Examples of capping enzymes include Vaccinia Capping Enzyme (VCE) (New England Biolabs, Ipswich, Mass.), a Bluetongue Virus capping enzyme, a Chiorella Virus capping enzyme, and a Saccharomyces cerevisiae capping enzyme.

In some embodiments, the method may include selecting for 3′ poly A tails. In some embodiments, poly A tail selection occurs by templated ligation, a poly A complementary reverse transcription, or poly A based hybridization capture.

In some embodiments, the method may further include selecting for capped RNA. In some embodiments, the method includes use of cap recognizing antibodies for immunoprecipitation. In some embodiments, the RNA non-capped RNAs are dephosphorylated prior to cap removal exposing a phosphate group.

In some embodiments, starting RNA samples may or may not undergo further manipulation steps prior to the enzymatic enrichment of capped RNAs set forth below. For example, if a large amount of degraded RNA is present in the starting RNA sample, the starting RNA sample can be treated with a polynucleotide kinase prior to enzymatic enrichment for capped RNAs to phosphorylate the 5′ end of degraded RNA. This phosphorylation will render RNA degradation fragments with 5′ OH groups sensitive to 5′ monophosphate-dependent exonuclease. Some embodiments further include phosphorylating the 5′ end of the RNA molecules with T4 PNK. In some embodiments, the enzymatic enrichment of capped RNA may improve enrichment of full-length RNA of at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, or ranges including and/or spanning the aforementioned values.

In some embodiments, the method only requires a small amounts of RNA, e.g., 5000 ng or less of starting RNA. In some embodiments, the starting RNA sample includes 10 ng, 20 ng, 25 ng, 30, ng 40 ng, 50 ng, 60 ng, 70 ng, 80 ng, 90 ng, 100 ng, 110 ng, 120 ng, 130 ng, 140 ng, 150 ng, 160 ng, 170 ng, 180 ng, 190 ng, 200 ng, 250 ng, 300 ng, 350 ng, 400 ng, 500 ng, 600 ng, 700 ng, 800 ng, 900 ng, 1000 ng, 1250 ng, 1500 ng, 1750 ng, 2000 ng 2500 ng, 3000 ng 3500 ng, 4000 ng, 4500 ng, 5000 ng, or ranges including and/or spanning the aforementioned values. In some embodiment, a starting RNA sample includes 5000 ng or less RNA, 2500 ng or less RNA, 1000 ng or less RNA, 750 ng or less RNA, 500 ng or less RNA, 250 ng or less RNA, or 100 ng or less RNA. In some embodiments, the method may start with less than 50 μg, 40 μg, 30 μg, 20 μg, 10 μg, 1 μg RNA, or ranges including and/or spanning the aforementioned values.

In some embodiments, the RNA sample includes at least 50 nucleotides, at least 75 nucleotides, at least 100 nucleotides, at least 150 nucleotides, at least 200 nucleotides, at least 250 nucleotides, at least 300 nucleotides, at least 350 nucleotides, at least 400 nucleotides, at least 450 nucleotides, at least 500 nucleotides, at least 550 nucleotides, at least 600 nucleotides, at least 650 nucleotides, at least 700 nucleotides, at least 750 nucleotides, at least 800 nucleotides, at least 850 nucleotides, at least 900 nucleotides, at least 950 nucleotides, at least 1000 nucleotides, at least 1100 nucleotides, at least 1200 nucleotides, at least 1300 nucleotides, at least 1400 nucleotides, at least 1500 nucleotides, at least 1600 nucleotides, at least 1700 nucleotides, at least 1800 nucleotides, at least 1900 nucleotides, at least 2000 nucleotides, at least 2100 nucleotides, at least 2200 nucleotides, at least 2300 nucleotides, at least 2400 nucleotides, at least 2500 nucleotides, at least 2600 nucleotides, at least 2700 nucleotides, at least 2800 nucleotides, at least 2900 nucleotides, at least 3000 nucleotides, at least 3100 nucleotides, at least 3200 nucleotides, at least 3300 nucleotides, at least 3400 nucleotides, at least 3500 nucleotides, at least 3600 nucleotides, at least 3700 nucleotides, at least 3800 nucleotides, at least 3900 nucleotides, at least 4000 nucleotides, at least 4100 nucleotides, at least 4200 nucleotides, at least 4300 nucleotides, at least 4400 nucleotides, at least 4500 nucleotides, at least 4600 nucleotides, at least 4700 nucleotides, at least 4800 nucleotides, at least 4900 nucleotides, at least 5000 nucleotides, at least 6000 nucleotides, at least 7000 nucleotides, at least 8000 nucleotides, at least 9000 nucleotides, at least 10,000 nucleotides, or ranges including and/or spanning the aforementioned values. In some embodiments, the RNA sample is between about 50 nucleotides and about 10000 nucleotides.

In some embodiments, the capped RNAs is in a degraded preparation. In one embodiment, a starting RNA sample includes degraded RNA.

In some embodiments, the exonuclease is a 5′ exonuclease. In some embodiments, the 5′ exonuclease is Terminator™ (Epicentre; TER51020). In some embodiments, the 5′ exonuclease is XRN-1 (New England Biolabs, Ipswich, Mass.). In some embodiments, the exonuclease degrades RNAs containing a 5′ monophosphate. In some embodiments, the exonuclease results in an apparent enrichment of primary transcripts containing 5′-triphosphates.

In some embodiments, the RNA sample is isolated from cells or tissue. RNA can be isolated from desired tissue or whole organisms (e.g., C. elegans). For example, the RNA sample may be isolated using RNeasy Universal Kit (Qiagen; Id:73404).

Often RNA samples are categorized as low quality or low RIN score. These samples are known to be difficult to prepare RNA libraries from and often contain fail long read library prep methods. In some embodiments, the RIN score is less than 10, less than 9, less than 8, less than 7, less than 6.5, less than 6, less than 5.5, less than 5, less than 4.5, less than 4, less than 3.5, less than 3, or ranges including and/or spanning the aforementioned values.

Some embodiments of the present disclosure improve yields and success rate in sequencing library generation from low quality RNA samples. In some embodiments, the improved yield is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or ranges including and/or spanning the aforementioned values. In some embodiments, the improved success rate is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or ranges including and/or spanning the aforementioned values.

In some embodiments, a population of molecules obtained using the method (e.g., RNA or cDNA) may be sequenced. In some embodiments, the RNA is sequenced directly. Illustrative non-limiting examples of nucleic acid sequencing techniques include, but are not limited to, chain terminator (Sanger) sequencing and dye terminator sequencing. Chain terminator sequencing uses sequence-specific termination of a DNA synthesis reaction using modified nucleotide substrates. In some embodiments, a next-generation sequencing technique may be used as alternatives to Sanger and dye-terminator sequencing methods (Voelkerding et al., Clinical Chem., 55: 641-658, 2009; MacLean et al., Nature Rev. Microbiol., 7: 287-296, 2009; each herein incorporated by reference in their entirety). In some embodiments, the sequencing may be pyrosequencing (Voelkerding et al., Clinical Chem., 55: 641-658, 2009; MacLean et al., Nature Rev. Microbiol., 7: 287-296; U.S. Pat. Nos. 6,210,891; 6,258,568). In some embodiments, the sequencing step may utilize a Solexa/Illumina platform ((Voelkerding et al., Clinical Chem., 55: 641-658, 2009; MacLean et al., Nature Rev. Microbiol., 7: 287-296; U.S. Pat. Nos. 6,833,246; 7,115,400; 6,969,488). In some embodiments, the sequencing step may utilize SOLiD technology (Voelkerding et al., Clinical Chem., 55: 641-658, 2009; MacLean et al., Nature Rev. Microbiol., 7: 287-296; U.S. Pat. Nos. 5,912,148; 6,130,073). In some embodiments, the sequencing step may include nanopore sequencing (see, e.g., Astier et al., J Am Chem Soc. 2006 Feb. 8; 128(5): 1705-10). In some embodiments, the RNA sequencing library may be prepared using a NEBNext™ Small RNA Library Prep kit. In some embodiments, third-generation sequencing platforms include those offered by Oxford Nanopore Technologies (ONT) and Pacific Biosciences (PacBio).

In some embodiments, the RNA may be converted to cDNA using reverse transcriptase enzymes. In some embodiments, the reverse transcriptase enzyme is a SuperScript™ IV Reverse Transcriptase (ThermoFisher). In some embodiments, the reverse transcriptase enzyme is a MMLV Reverse Transcriptase (ThermoFisher). In some embodiments, the reverse trnasciptase is an Avian myeloblastosis virus (AMV) reverse transcriptase. In some embodiments, the reverse transcriptase is a GoScript™ Reverse Transciptase (Promega). In some embodiments, the reverse transcriptase is a Maxima™ H Minus Reverse Transcriptase (ThermoFisher). In some embodiments, the reverse transcriptase is a MarathonRT Reverse Transcriptase (From the laboratory of Anna Marie Pyle, PhD, Yale University). In some embodiments, the reverse transcriptase is a NEBNext™ Single Cell Reverse Transcriptase (New England Biolabs).

Some embodiments further include using template switch oligos and compatible enzymes to convert the RNA to cDNA. In some embodiments, the temple switch oligo is a chimeric DNA/RNA oligo. In some embodiments, the template switch oligo is from about 20 to about 100 bases.

In some embodiments, the RNA is first ligated with a reverse transcription adapter.

In some embodiments, a method described herein may further comprise adding a poly(A) tail to the RNA. In these embodiments, the method may include eluting the enriched RNA from the affinity matrix; making cDNA from the enriched RNA in the presence of a template switching oligonucleotide, using an oligo(dT) primer that hybridizes to the poly(A) tail, wherein the reverse transcriptase used to make the cDNA switches templates from an RNA molecule to the template switching oligonucleotide during cDNA synthesis to produce cDNAs that contains a 5′ end having the sequence of oligo(dT) primer and a 3′ end containing the reverse complement of the template switching oligonucleotide; and sequencing the cDNA.

In these embodiments, the method may further include adding a 3′ poly(A) tail to the RNA where the RNA molecules do not otherwise have a poly(A) tail (e.g., as is the case for most prokaryotic RNA, and some eukaryotic RNA molecules including fragmented eukaryotic mRNA that can be enriched using this method); and/or enriching for poly(A) RNA using an affinity matrix that binds to poly(A). In some embodiments, the method may further include amplifying the cDNA using primers that hybridize with the 3′ end and the 5′ end of the cDNA. In some embodiments, the amplifying the cDNA using primers that hybridize with the 3′ end and the 5′ end of the cDNA occurs after making cDNA from the enriched RNA and before sequencing the cDNA.

### Kits

Also provided by this disclosure are kits for practicing the methods as described herein. A kit may contain an exonuclease and reverse transcription reaction materials comprising at least one of a buffer, primers, dNTP mix, and a reverse transcriptase, or a combination thereof. In some embodiments, a kit may contain unconjugated oligos, kinases, reverse transcription enzymes, 5′ exonucleases, RNA cleanup reagents and/or columns.

The components of the kit may be combined in one container, or each component may be in its own container. For example, the components of the kit may be combined in a single reaction tube or in one or more different reaction tubes. Further details of the components of this kit are described above. The kit may also contain other reagents described above and below that are not essential to the method but nevertheless may be employed in the method, depending on how the method is going to be implemented.

In addition to above-mentioned components, the subject kits may further include instructions for using the components of the kit to practice the subject methods, i.e., to provide instructions for sample analysis. The instructions for practicing the present method may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1

In this example, an embodiment of a method for enriching RNA is described.

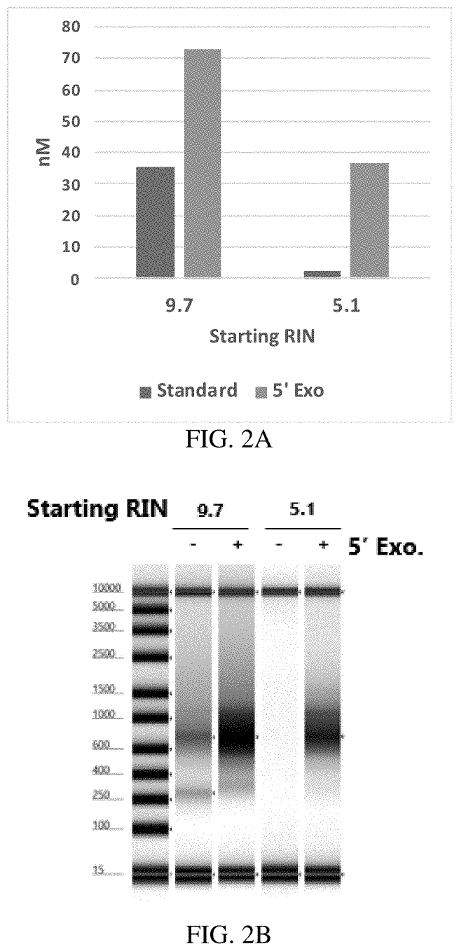

To determine if RNA samples with differing RIN scores would benefit from enrichment using treatments with a 5′ exonuclease, a sample with a starting RIN of 9.7 and a sample with a starting RIN of 5.1 was contacted with 10.5 units of PNK for 30 minutes. The sample is then cleaned using a Zymogen Clean and Concentrator™ and treated with 9 units of the Terminator™ exonuclease and incubated for 75 minutes. Following final treatment, the RNA is again cleaned, and the concentration is determined. 50 ng of RNA for each sample where then treated with an oligodT primers and reverse transcriptase to calculate how much cDNA would be obtained from each sample. FIG. 2A shows the results of this experiment wherein the RIN 9.7 sample had over 70 ng of cDNA obtained after Terminator™ treatment as compared to about 35 ng of cDNA without Terminator™ treatment. The much lower quality sample, having a RIN of 5.1 was able to obtain almost 40 ng of cDNA after Terminator™ treatment, whereas without Terminator™ treatment almost no cDNA was able to be amplified from the degraded sample. FIG. 2B shows that the majority of the cDNA amplified in the samples treatment with Terminator™ were approximately 600-1000 nt in length, whereas the samples without treatment with Terminator™ had little to no cDNA in these size ranges.

This example shows that enrichment with Terminator™ followed by reverse transcription using an oligodT primer can greatly increase the cDNA yields from degraded RNA samples.

### Example 2

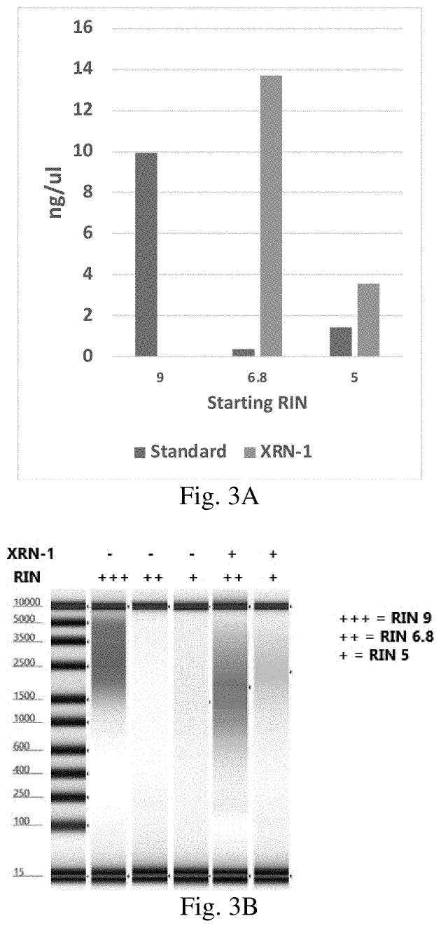

FIGS. 3A and 3B show an example of an experiment similar to the above, except using an XRN1 nuclease in place of Terminator™ on samples having a RIN score of 9, 6.8 or 5. Additional changes include using 300 ng RNA after XRN1 treatment and performing the library prep following PacBio protocols. As shown, the XRN1 treatment greatly improved the amount and size of the cDNA obtained from the RNA samples.

### Example 3

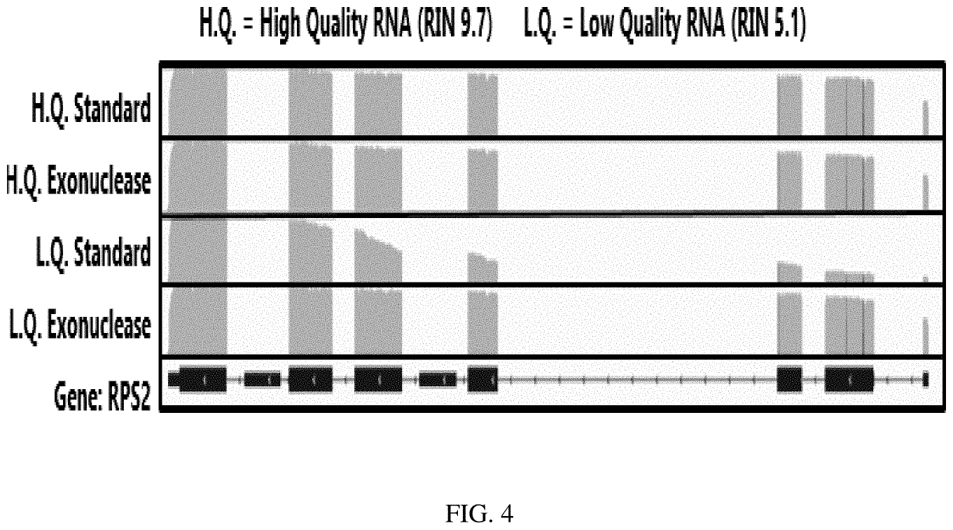

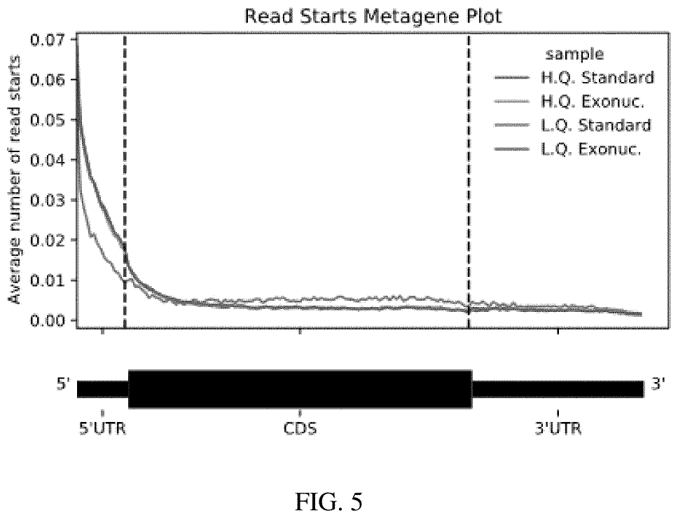

FIG. 4 shows the results of Example 1, displayed to show the quality of the cDNA that was derived from the experiment. The cDNA corresponded to the RPS2 gene shown along the bottom line of the chart in FIG. 4. The chart shown the number of cDNA molecules corresponding to each exon of the RPS2 gene, indicating that the cDNA which is at the furthest right side of the figure is full length cDNA which includes all of the exons shown along the bottom line of the chart. As shown, the cDNA derived from the high quality RIN 9.7 samples, with or without exonuclease treatment included all of the exons in the RPS2 gene and thus were mostly full-length. However, the cDNA derived from the RIN 5.1 sample and untreated with exonuclease did not include much of the 3′ exons to the right side of the chart. In comparison, the cDNA derived from the RIN 5.1 sample and treated with exonuclease resembled the high quality RIN 9.7 results, showing that the cDNA included all of the exons within the RPS2 gene.

This confirms that treatment with exonuclease, and reverse transcription using oligodT primers, even in a sample with a RIN score of 5.1, can recover and enrich for full-length RNA samples.

### Example 4

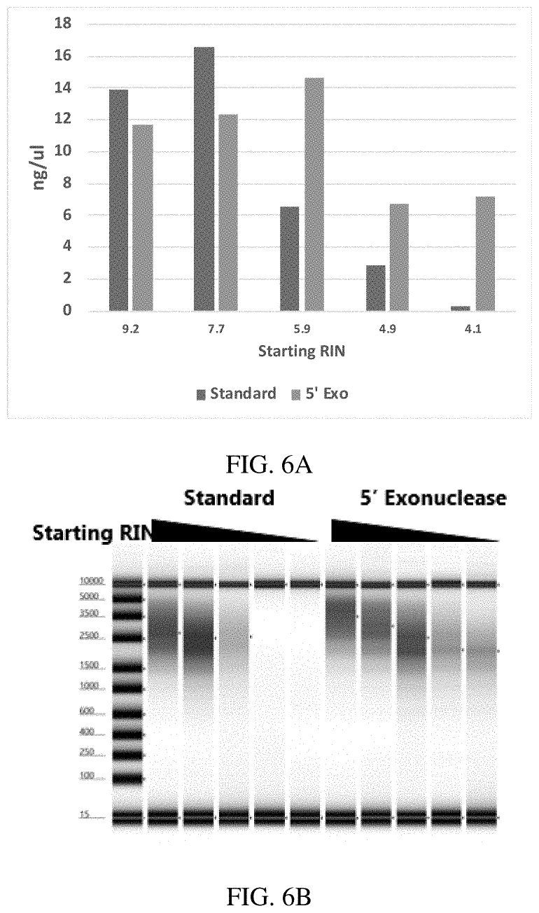

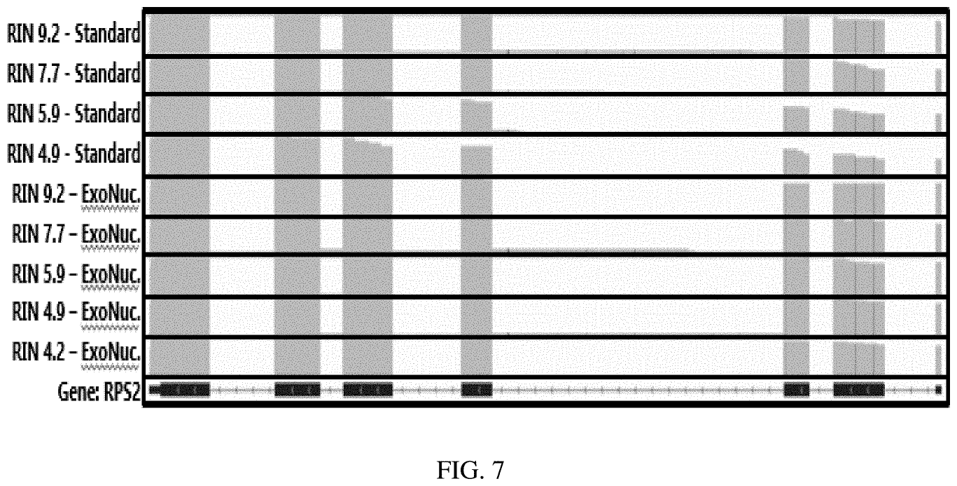

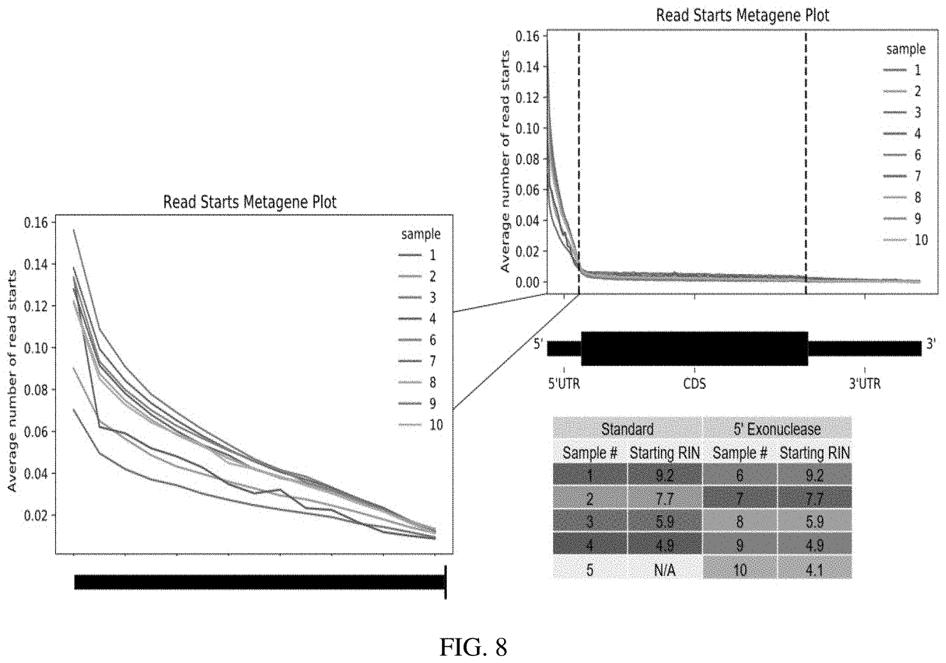

In this example, the protocols described herein were similar to example 2 but with the use of Terminator instead of XRN-1 (FIG. 6A and FIG. 6B). The genome track view of RPS2 showing improved coverage across the gene with exonuclease treatment with PacBio Isoseq library preperation (FIG. 7). It was determined that a transcriptome wide metagene plot of the 5′ end of reads with PacBio Isoseq library preperation produced more full length reads upon Terminator treatment (FIG. 8).

### Example 5-Alternative 5′ Selection

Use an antibody recognizing the 5′ cap structure and perform an immunoprecipitation to isolate RNA molecules containing the cap structure.

### Example 6-Alternative 5′ Selection

Dephosphorylate all available 5′ ends on a RNA sample. Cap containing molecules are protected from dephosphorylation. Following dephosphorylation, 5′ cap is removed enzymatically leaving a 5′ phosphate only on molecules that originally contained cap. Oligos are then ligated to RNAs containing a 5′ phosphate.

Although described in some detail for purposes of illustration and clarity, it will be readily appreciated from a reading of this disclosure that various changes in form and detail that are known or appreciated by those of skill in the art may be practiced without departing from the true scope of the disclosure. For example, all the techniques and apparatus described above can be used in various combinations, e.g., sequentially or simultaneously. All terms used herein are intended to have their ordinary meaning unless an alternative definition is expressly provided or is clear from the context used therein. To the extent any definition is expressly stated in a patent or publication that is incorporated herein by reference, such definition is expressly disclaimed to the extent that it is in conflict with the ordinary meaning of such terms, unless such definition is specifically and expressly incorporated herein, or it is clear from the context that such definition was intended herein. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all publications, patents, patent applications, and/or other documents referred to in this disclosure are incorporated herein by reference in their entireties for all purposes to the same extent as if each individual publication, patent, patent application, and/or other document were individually and separately indicated to be incorporated by reference for all purposes.

What is claimed is:

1. A method of enriching for full length RNA, comprising:
contacting an RNA sample with a 5′ exonuclease; and
selecting for 3′ poly A tails.

2. The method of claim 1, wherein the poly A tail selection occurs by templated ligation, a poly A complementary reverse transcription, or poly A based hybridization capture.

3. The method of claim 1, wherein the RNA sample is derived from a source selected from the group consisting of: cells, tissue, FFPE, blood, urine, and saliva.

4. The method of claim 1, wherein the RNA sample comprises 5 μg or less of RNA.

5. The method of claim 1, wherein the RNA sample comprises at least one of degraded RNA, partially degraded RNA, capped RNA, and mRNA molecules.

6. The method of claim 1, wherein the RNA sample is rRNA depleted.

7. The method of claim 1, further comprising sequencing the full length RNA.

8. The method of claim 1, further comprising performing a reverse transcription reaction to convert the RNA to cDNA and sequencing the cDNA.

9. The method of claim 1, wherein the RNA sample has a starting RIN score of less than 7.

10. The method of claim 8, wherein sequencing of the cDNA is further subjected to a size selection procedure to produce a cDNA library.

11. The method of claim 10, further comprising amplifying the cDNA library by third generation sequencing technologies.

12. The method of claim 1, further comprising selecting for capped RNA.

13. The method of claim 12, further comprising use of cap recognizing antibodies for immunoprecipitation and dephosphorylation of non-capped RNAs followed by cap removal.

14. A method of enriching for full length RNA, the method comprises:
isolating an RNA sample;
contacting the RNA sample with a 5′ exonuclease;
selecting for 3′ poly A tails; and performing a reverse transcription reaction to convert the RNA to cDNA,
thereby enriching for capped RNA for full length RNA reads.

15. The method of claim 14, wherein the RNA sample comprises at least one of degraded RNA, partially degraded RNA, mRNA molecules, and capped RNA.

16. The method of claim 14, wherein the RNA sample is rRNA depleted.

17. The method of claim 14, wherein the poly A tail selection occurs by templated ligation, a poly A complementary reverse transcription, or poly A based hybridization capture.

18. The method of claim 14, wherein an alternative 5′ cap selection method is used, including the use of cap recognizing antibodies for immunoprecipitation and dephosphorylation of non-capped RNAs followed by cap removal.

19. The method of claim 14, wherein the method improves yields of low quality RNA by at least 70%.

20. A kit comprising:
a first component comprising a 5′ exonuclease;
a second component comprising reverse transcription reaction materials comprising buffer, primers, dNTP mix, and a reverse transcriptase; and
a manual providing instructions for enriching full length RNA.
